(19) United States
(12) Patent Application Publication
(10) Pub. No.: US 2004/0054178 A1
(43) Pub. Date: Mar. 18, 2004

(54) 1,7, and 1,9-diarylpolymethine salts

(76) Inventors: Yves Madaule, Rebigue (FR); Corrine Payrastre, Toulouse (FR); Albert Izquierdo, Toulouse (FR)

Correspondence Address: BURNS DOANE SWECKER & MATHIS L L P, POST OFFICE BOX 1404, ALEXANDRIA, VA, 22313-1404, US

(21) Appl. No.: 10/433,890

(22) PCT Filed: Dec. 5, 2001
(86) PCT No.: PCT/FR01/03844

(30) Foreign Application Priority Data
Dec. 7, 2000 (FR) 00/15928
Jul. 20, 2001 (FR) 01/09743

(51) Int. Cl. C07F 9/02; C07D 239/02

(52) U.S. Cl. 544/242; 564/12; 564/230; 564/225; 564/251; 564/336

(57) ABSTRACT

The invention relates to 1,7-diarylpentamethine and 1,9-diarylheptamethine salts, in partricular heptacarbon or nonacarbon carboxonium salts, and streptocyanines.

The compounds correspond to formula [G-D-G′]⁺Q⁻ in which Q is an anion of a strong acid, and

G and G′ represent, independently of each other, an OEt, amino, hydrazono, hydrazino, phosphaimino, amidino or guanidino group; or a multivalent radical optionally linked at at least one of its other ends to another group D;

D represents a cationic group 1,7-diarylpentamethine or 1,9-diarylheptamethine in which the aryl groups carry substituents representing, independently of each other, a hydrogen, a halogen, an alkyl radical or an alkyloxy radical having from 1 to 15 carbon atoms or an acetamido group CH₃C(O)HN—.

The present invention relates to heptacarbon or nonacarbon carboxonium salts and streptocyanines, their method of preparation, and their use as biological markers.

Chemical, chromatographic or spectroscopic methods have for long not been very well suited to the detection of molecules in the nano- and subnanogram range, because of inadequate sensitivity.

Since the 1960s, radioisotope labeling, and in particular the use of radioactive iodine (¹²⁵I), appeared to be the analytical method of choice for the detection of endogenous and exogenous substances (hormones, bacteria, viruses, toxins, and the like). This currently commonly used technique is nevertheless tending to disappear. Novel labeling techniques have been studied and the technological improvement in the equipment used in spectroscopic methods has caused radioactive methods of labeling to be gradually abandoned. The latter have been replaced by organic molecules termed fluorophores, in particular by cyanines. Cyanines are compounds which are used for coupling with biological molecules and can be used as markers because of their triple character: positively charged, lipophilic and fluorescent. It is known to use certain cyanines as markers, in combination with antibodies, DNA, proteins, polysaccharides and other biological molecules, for assaying, monitoring active substances in vivo and in vitro or for the diagnosis of various diseases. For a compound to be used as a biological marker, it should have an absorption and emission domain shifted to the near-infrared so as not to interfere with the substrate autofluorescence region. It should also be able to be grafted onto the target molecule by covalent bonding or by complexation.

Various methods for preparing cyanines are known. The method described for example by S. R. Mujumdar, et al., [“Cyanine-Labeling Reagents: Sulfobenzindocyanines Succinimidyl Esters”, Bioconjugate Chem . 1996, 7, 356-362], consists in reacting triethyl orthoformate (or triethoxymethane) with 2-methylindole derivatives in order to obtain cyanines which have three methine groups between the terminal indole groups and which have a maximum absorption wavelength (λₘₐₓ) of the order of 580 nm. Cyanines of this type, called Cy3, are marketed by the company Amersham Life Science. The reaction of 1,3,3-trimethoxypropene (instead of triethoxymethane) with an activated 2-methylindole type derivative in order to obtain cyanines which have five methine groups between the terminal indole groups and which have a wavelength λₘₐₓ of the order of 680 nm have been described by the same authors. Cyanines of this type, called Cy5, are also marketed by the company Amersham Life Science.

Another method for preparing streptocyanines is described by C. Payrastre et al. [“A Synthetic Pathway to Macrocyclic and Optically Active Pentamethinium Salts” Tetrahedron Letters 1994,35(19), 3059-3062]. It consists in reacting a pentacarbon carboxonium salt with a primary amine or a secondary amine, and then, by extension, with phosphaimines, amidines, guanidines, hydrazines or hydrazones. The pentacarbon carboxonium salt is obtained by reaction of an aryl methyl ketone with triethoxymethane and perchloric acid. This method is relatively simple to carry out. The streptocyanines obtained have nevertheless a wavelength λₘₐₓ which remains less than a value of the order of 600 nm, which limits their use as a marker in the near-infrared.

According to another method, cyanines can be obtained by condensing a heterocyclic base containing an activated methyl group and a bisaldehyde or any other equivalent of the Schiff base type in the prsence or otherwise of a catalyst. The diversity of existing heterocyclic bases offers a practically infinite choice for the preparation of cyanines. However, only two types of bisaldehydes are currently known which are capable of being used for the synthesis of nonacarbon cyanines.

The first type of bisaldehyde is a glutaconaldehyde salt corresponding to the formula:

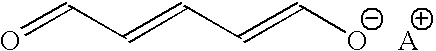

The corresponding Schiff base corresponds to the formula:

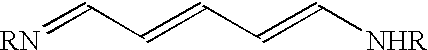

It makes it possible to obtain various types of linear cyanines, without any functionalization on the polymethine chain, as described for example by Y. Nagao, et al., [“Synthesis and Reactivities of 3-Indocyanine-green-acyl-1,3-thiazolidine-2-thione-(ICG-ATT) as a New Near-Infrared Fluorescent-labeling Reagent”, Bioorganic and Medicinal Chemistry, 1998, 6, 2179-2184] and by A. S. Waggoner, et al., [“Cyanine Dye Labeling Reagents for Sulfhydryl Groups”, Cytometry. 1989, 10, 3-10].

The second bisaldehyde is of the 2-Q-1-formyl-3-hydroxy-methylenecyclohexene type in which Q is most often a hydrogen or a chlorine [G. A. Reynolds, and K. H. Drexhage, “Stable Heptamethine Pyrylium Dyes that Absorb in the Infrared”, J. Org. Chem. 1977 Vol. 42, No. 5, 885-888].

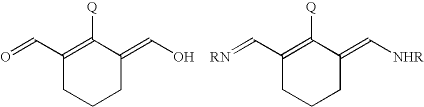

Unlike the derivatives of the glutaconaldehyde salt, the 2-chloro-1-formyl-3-hydroxymethylenecyclohexene derivative allows functionalization of the polymethine chain but also rigidification of the system [G. Patonay, et al., “Functionalization of Near-Infrared Cyanine Dyes”, J. Heterocyclic Chem. 1996, 33, 1685] or [N. Narayanan, et al., “A New Method for the Synthesis of Heptamethine Cyanine Dyes: Synthesis of New Near-Infrared Fluorescent Labels”, J. Org. Chem. 1995, 60, 2391-2395].

The aim of the present invention is to provide novel functionalized cyanines having a high absorption wavelength, which can be used in particular as biological markers.

Accordingly, the subject of the invention is salts in which the cation comprises a 1,7- or 1,9-diarylpolymethine group, and a method for their preparation, and their use as biological markers.

A compound according to the invention corresponds to the following formula (I):

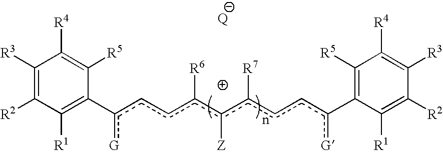

in which:

Q⁻ is an anion of a strong acid;

n is 0 or 1;

G and G′ represent, independently of each other, an OEt group, an amino group, a phosphaimino group, an amidino group, a guanidino group, a hydrazino group, a hydrazono group, or a multivalent radical linked at at least one of its other ends to a radical corresponding to formula (I′) below

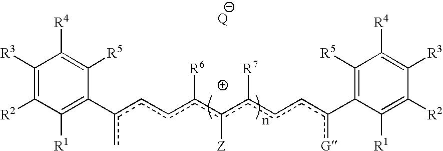

in which G″ represents an OEt group, an amino group, a phosphaimino group, an amidino group, a guanidino group, a hydrazino group, a hydrazono group, or a multivalent radical;

R¹ to R⁵ represent, independently of each other, a hydrogen, a halogen, an alkyl radical, an alkyloxy radical having from 1 to 15 carbon atoms or an acetamido group CH₃C(O)HN—;

Z represents H or a halogen,

R⁶ and R⁷ represent, independently of each other, H, or alternatively R⁶ and R⁷ form together a 3- or 4-membered biradical optionally carrying one or more substituents chosen from methyl or ester groups, it being understood that R⁶ represents H when n=0.

The anion is preferably chosen from BF₄⁻, CF₃SO₃^{-, ClO}₄⁻, I⁻, Br⁻ and Cl⁻.

When G, G′ or G″ represents a multivalent radical, it is preferably chosen from the groups —NH-E-NH— in which E is —(CH₂)ₙ—, 3≦n≦9, or —(CH₂)₂O(CH₂)₂O(CH₂)₂—.

Among the compounds of the present invention, those which correspond to formula (II) below are particularly advantageous, in particular because they make it possible to obtain the other compounds (I).

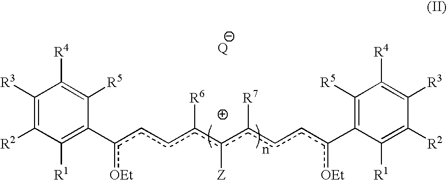

In formula (II), R¹ to R⁷, n, Q and Z have the meaning given above, and Et represents an ethyl group.

When n=0 and R⁶ represents H, they correspond to the following formula (II_{A}).

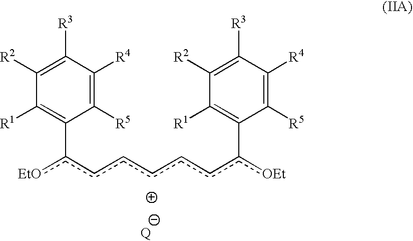

When n=1, they correspond to the following formula (II_{B}).

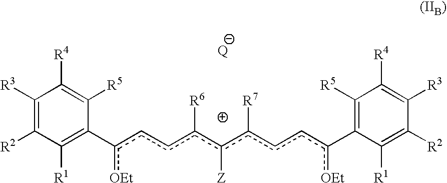

A compound (II_{A}) according to the present invention may be prepared from an aryl ketone Ar—C(O)R′ (designated below by AK) in which Ar represents a phenyl radical carrying the substituents R¹ to R⁵ defined above and R′ represents an alkyl radical having from 1 to 5 carbon atoms, preferably a methyl.

The method according to the invention for the preparation of a compound (II_{A}) is characterized in that it consists in reacting the aryl ketone (AK) with a mixture of triethoxymethane (TEM) and 1,3,3-triethoxypropene (TEP) in the presence of a strong acid, under an inert atmosphere, in anhydrous medium, at a temperature between −5° C. and 80° C., using quantities of reagents such that the mol ratios are the following: 0.25≦TEP/TME≦3 and 1/4≦AK/TEM+TEP≦2.

The inert atmosphere is advantageously obtained by carrying out the procedure under argon. The method is preferably carried out at room temperture. The TEP/TEM ratio is preferably equal to 1 and the AK/TEM+TEP ratio is preferably equal to 1 in order to avoid the formation of undesirable by-products.

Depending on the nature of the anion Q, the strong acid is chosen from HBF₄, CF₃SO₃H, HClO₄, HI, HBr or HCl.

The compound obtained in the reaction medium may be recovered by precipitation, filtration, washing and drying. The precipitation may be performed in a solvent such as an ether, a hydrocarbon or a nonpolar solvent. By way of example, there may be mentioned ethyl ether, THF, pentane, hexane, cyclohexane, cyclopentane or carbon tetrachloride.

The reaction is illustrated by the following scheme, corresponding to the specific case of 4-methylacetophenone:

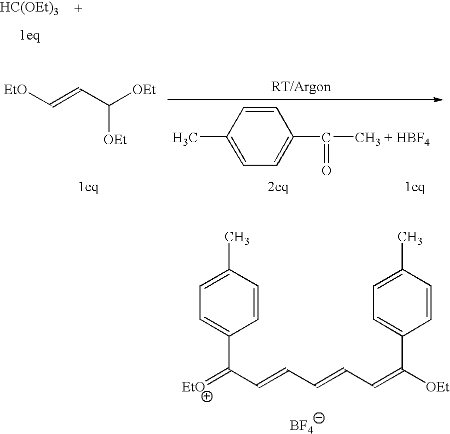

Trials to prepare a compound (II_{A}) from an aryl ketone had been made by the inventors by replacing the triethoxymethane (used for the preparation of a pentacarbon 1,5-diarylcarboxonium salt according to the prior art) with triethoxypropene. However, these trials did not make it possible to obtain the expected compound (II_{A}). It appeared that there was being formed in particular a pyrylium salt and that a large portion of the triethoxypropene was irreversibly hydrolyzed in the reaction medium, according to the following scheme:

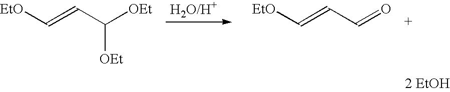

The inventors then found that, surprisingly, addition of triethoxymethane to the reaction medium made it possible to obtain the expected compound (II_{A}), with, as a by-product, the pyrylium salt (when TEM/TEP<1) or the pentacarbon carboxonium salt (when TEM/TEP>1).

The triethoxymethane is a compound which is commercially available under the name ethyl orthoformate.

1,3,3-Triethoxypropene can be prepared by the method described by M. Lounasmaa, et al., [Tetrahedron Letters, 1995, Vol. 51, No. 31, pp. 8623-8648]. This method consists in reacting acrolein with bromine in order to obtain 2,3-dibromopropionaldehyde, which is then converted to 2-bromo-3-ethoxypropionaldehyde diethyl acetal by reaction with EtOH/HCl or EtOH/para-toluenesulfonic acid. This compound is refluxed in ethanol in the presence of KOH and a Z and E 1,3,3-triethoxypropene mixture is obtained.

A compound (II_{B}) according to the present invention may be prepared from an aryl ketone ArC(O)R′ (designated below by AK) in which Ar represents a phenyl radical carrying the substituents R¹ to R⁵ defined above and R′ represents an alkyl radical having from 1 to 5 carbon atoms, preferably a methyle. The method for preparing a compound (II_{B}) is characterized in that it consists in reacting aryl ketone (AK) with a mixture of triethoxymethane (TEM) and a bisaldehyde (BA) in the presence of a strong acid, under an inert atmosphere and in an anhydrous medium. The temperature is preferably between −5° C. and 80° C., and the quantities of reagents are such that the mol ratios are the following: 1/6≦BA/TEM<1/3 and 2/7≦AK/TEM+BA<0.5.

Bisaldehyde corresponds to formula (A)

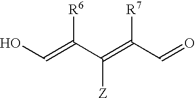

in which the substituants Z, R⁶ and R⁷ have the meaning given above. By way of example, there may be mentioned 2-chloro-1-formyl-3-hydroxymethylenecyclohexene (CFHMCH), 1-formyl-3-hydroxymethylenecyclohexene (FHMCH), 2-chloro-1-formyl-3-hydroxymethylenecyclopentene (CFHMCP), 1-formyl-3-hydroxymethylenecyclopentene (FHMCP), and a glutaconaldehyde salt. CFHMCH is marketed by the company Aldrich (CAS No.: 61010-04-6).

The inert atmosphere is advantageously obtained by carrying out the procedure under argon. The method is preferably carried out at room temperature. The BA/TEM ratio is preferably equal to 1/4 and the AC/TEM+BA ratio is preferably equal to 2/5 in order to limit the formation of undesirable by-products. Depending on the nature of the anion Q, the strong acid is chosen from HBF₄, CF₃SO₃H, HClO₄, HI, HBr or HCl.

The compound (II_{B}) obtained in the reaction medium may be recovered by precipitation, filtration, washing and drying. The precipitation may be performed in a solvent such as an ether, a hydrocarbon or a nonpolar solvent. By way of example, there may be mentioned ethyl ether, THF, pentane, hexane, cyclohexane, cyclopentane or carbon tetrachloride.

The reaction is illustrated by the following scheme, corresponding to the specific case of 4-methylacetophenone and CFHMCH, in the presence of tetrafluoroboric acid:

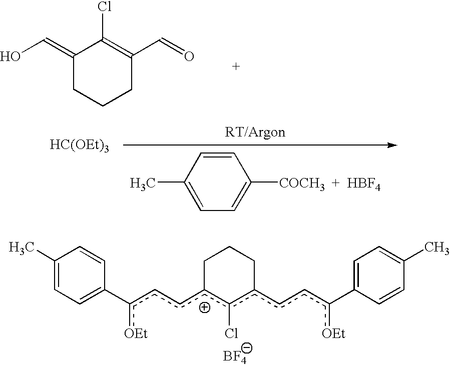

The use of the method for preparing a compound (II_{B}) described above by reacting 4-methylacetophenone and CFHMCH), but omitting the use of triethoxymethane, did not make it possible to obtain the expected compound (II_{B}). There is formed in particular a diketone compound corresponding to the following formula (II_{B}′)

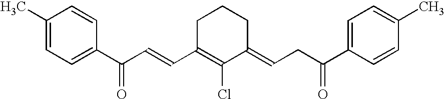

A compound according to the invention may additionally be a streptocyanine corresponding to formula (III) below:

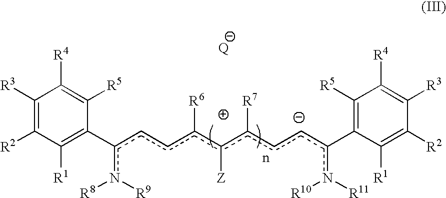

in which:

R¹ to R⁷, n, Q and Z have the meaning given above;

R⁸, R⁹, R¹⁰ and R¹¹ are chosen, independently of each other, from:

H;

alkyl radicals having from 1 to 12 carbon atoms;

phenyl radicals optionally carrying substituents chosen, independently of each other, from H, halogens, alkyl or alkyloxy radicals having from 1 to 15 carbon atoms or the acetamido group CH₃C(O)HN;

the groups —N═CHA′ and —NHA′ in which A represents a phenyl group optionally carrying one or more alkyloxy or dialkylamine substituents, it being understood that when R⁸ (respectively R¹⁰) is an —N═CHA′ and —NHA′, R⁹ (respectively R¹¹) is a methyl group.

or alternatively R⁸ and R⁹ and/or R¹⁰ and R¹¹ form together an aliphatic ring optionally comprising an oxygen atom.

The streptocyanines (III) are symmetrical when the pairs of substituents (R⁸, R⁹) and (R¹⁰, R¹¹) are identical.

The heptacarbon streptocyanines of the (III) type are represented by the following formula (III_{A}):

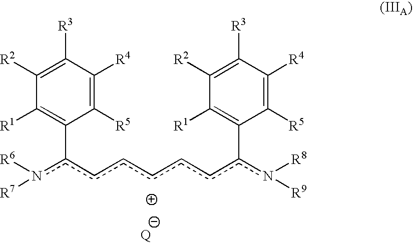

The nonacarbon streptocyanines are represented by the following formula (III_{B}):

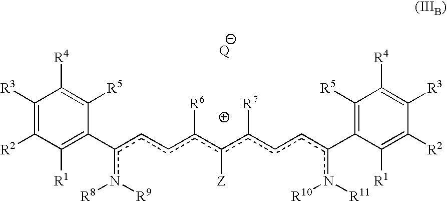

A compound of the invention may also be a streptocyanine corresponding to the following formula (IV):

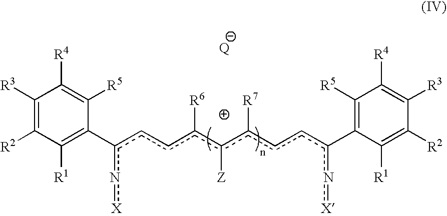

in which:

R¹ to R⁷, n, Q and Z have the meaning given above;

X and X′ represent, independently of each other, R″₃P, R″₂N(R′)C, (R″₂N)₂C or NR″₂, R″ representing an alkyl preferably having from 1 to 4 carbon atoms, or a phenyl.

The streptocyanines (IV) are symmetrical when the substituents X and X′ are identical.

A heptacarbon streptocyanine of the (IV) type is represented by the following formula (IV_{A}):

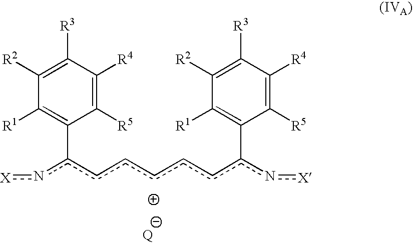

A nonacarbon streptocyanine of the (IV) type is represented by the following formula (IV_{B}):

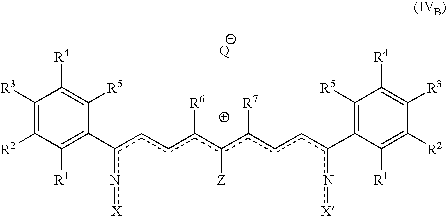

A compound of the presnt invention may also be a macrocyclic dicationic compound corresponding to the following formula (V):

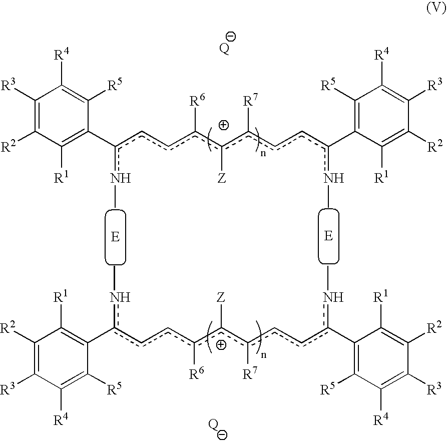

in which R¹ to R⁷, n, Q and Z have the meaning given above, and E is a spacer group preferably chosen from —(CH₂)ₙ— with n=3 to 9 or —(CH₂)₂O(CH₂)₂O(CH₂)₂—.

A dicationic macrocyclic compound (V) in which each cationic group is heptacarbon-based corresponds to the following formula (V_{A}):

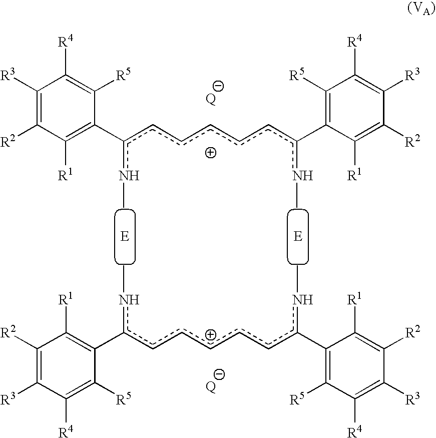

A dicationic macrocyclic compound (V) in which each cationic group is nonacarbon-based corresponds to the following formula (V_{B}):

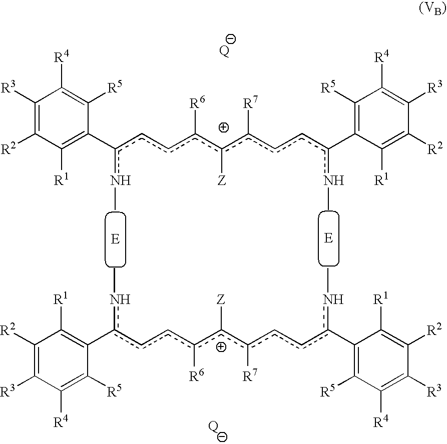

A compound according to the invention may be a diaryl hemicarboxonium salt corresponding to the following formula (VI):

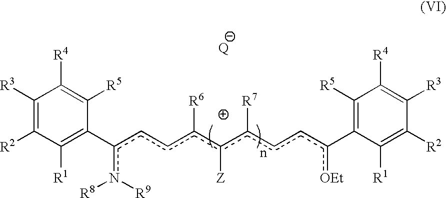

in which the various substituents have the meaning given above.

A heptacarbon (VI) type salt corresponds to the following formula (VI_{A})

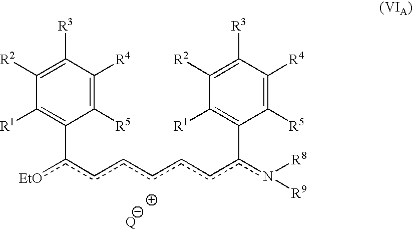

A nonacarbon (VI) type salt corresponds to the following formula (VI_{B}):

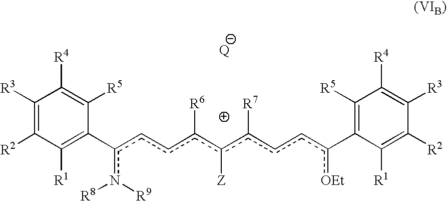

A compound according to the invention may additionally be a diaryl hemicarboxonium salt corresponding to the following formula (VII):

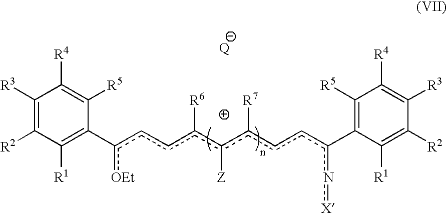

in which the various substituents have the meaning given above.

A heptacarbon (VII) type salt corresponds to the following formula (VII_{A}):

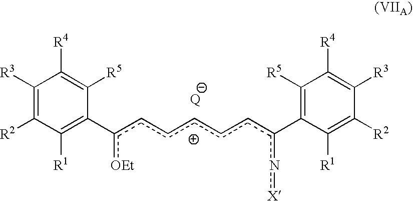

A nonacarbon (VII) type salt corresponds to the folowing formula (VII_{B}):

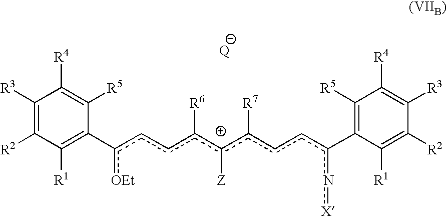

A compound according to the invention may also be a nonmacrocyclic polycationic compound (VIII) when one of the substituents G or G′ is a multivalent group linked at each of its ends to a group corresponding to formula (I′) defined above. The multivalent group is preferably a group —NH—(CH₂)ₙ—NH— with n=3 to 9 or a group —NH—(CH₂)₂O(CH₂)₂O(CH₂)₂—NH—.

The method for preparing a symmetrical streptocyanine (III) of the invention consists in reacting a salt (II) with a nitrogen-containing compound, using at least two equivalents of a nitrogen-containing compound per one equivalent of salt, said nitrogen-containing compound being chosen fron amines, hydrazines and hydrazones. The use of a heptacarbon salt (II_{A}) makes it possible to obtain a streptocyanine corresponding to formula (III_{A}). The use of a nonacarbon salt (II_{B}) makes it possible to obtain a streptocyanine corresponding to formula (III_{B}).

The method for preparing a symmetrical streptocyanine (IV) of the invention consists in reacting a compound (II) with a nitrogen-containing compound, using at least two equivalents of nitrogen-containing compound per one equivalent of salt, said nitrogen-containing compound being chosen from guanidines, phosphaimines and amidines. The use of a heptacarbon salt (II_{A}) makes it possible to obtain a streptocyanine corresponding to formula (IV_{A}). The use of a nonacarbon salt (II_{B}) makes it possible to obtain a streptocyanine corresponding to formula (IV_{B}).

A macrocyclic dicationic compound (V) is obtained by reacting a compound (II) with a diamine H₂N-E-NH₂, using a (II)/diamine molar ratio of 1/1. The use of a heptacarbon salt (II_{A}) makes it possible to obtain a streptocyanine corresponding to formula (V_{A}). The use of a nonacarbon salt (II_{B}) makes it possible to obtain a streptocyanine corresponding to formula (V_{B})The method for preparing a hemicarboxonium salt (VI) or (VII) consists in reacting a compound (II) with a nitrogen-containing compound, using one equivalent of nitrogen-containing compound per one equivalent of compound (II). The nitrogen-containing compound is chosen from amines, hydrazines, hydrazones for a compound (VI) or from guanidines, phosphaimines and amidines for compounds (VII). The use of a heptacarbon salt (II_{A}) makes it possible to obtain a heptacarbon hemicarboxonium salt corresponding to formula (VI_{A}) or (VII_{A}), respectively. The use of a nonacarbon salt (II_{B}) makes it possible to obtain a nonacarbon hemicarboxonium salt corresponding to formula (VI_{B}) or (VII_{B}) respectively.

A hemicarboxonium salt (VI) may be advantageously used for the preparation of disymmetrical streptocyanines (III), by reacting one equivalent of compound (VI) with one equivalent of a nitrogen-containing compound chosen from amines, hydrazines, hydrazones different from that used for the prepration of said compound (VI) from compound (II). The use of a salt (VI_{A}) makes it possible to obtain a disymmetrical cyanine (III_{A}), whereas the use of a salt (VI_{B}) makes it possible to obtain a disymmetrical streptocyanine (III_{B}).

A hemicarboxonium salt (VII) may be advantageously used for the preparation of disymmetrical streptocyanines (IV), by reacting one equivalent of compound (VII) with one equivalent of a nitrogen-containing compound chosen from guanidines, phosphaimines and amidines different from that used for the preparation of said compound (VII) from compound (II). As above, the use of a salt (VII_{A}) makes it possible to obtain a disymmetrical streptocyanine (IV_{A}), whereas the use of a salt (VII_{B}) makes it possible to obtain a disymmetrical streptocyanine (IV_{B}).

A hemicarboxonium salt (VI) may additionally be used for the preparation of nonmacrocyclic dicationic compounds (VIII), by reacting n equivalents of salt (VI) with one equivalent of a primary or secondary polyamine. Thus, di-, tri-, tetra- or polycationic compounds may be obtained by reacting two, three, four or n equivalents (n>4) of salt (VI) with a diamine, a triamine, a tetramine or a polyamine, respectively.

The hemicarboxonium salts (VI) may be grafted onto a substrate carrying nitrogen-containing functional groups, via the OEt functional group.

The streptocyanines (III_{B}) of the invention in which Z is a halogen may be functionalized by replacing the halogen atom by a group -M-φ-A. The compound then corresponds to formula

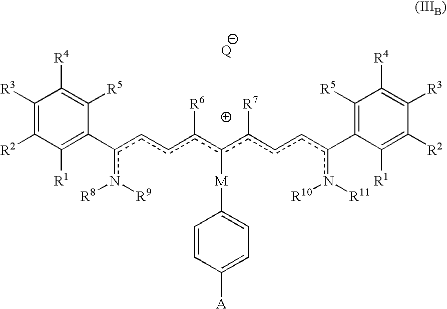

in which M may be an oxygen or sulfur atom, and A may be —NH₂ or an isothiocyanato group —N═C═S.

The streptocyanines of the present invention corresponding to formulae (III), (IV), (VI) or (VII) may be advantageously used as markers for various biological molecules such as for example antibodies, DNA, proteins and polysaccharides.

Another subject of the present invention is a method for labeling biological molecules, characterized in that it uses a streptocyanine according to the present invention.

The present invention is illustrated in greater detail with the aid of a few examples to which it is nevertheless not limited.

The bisaldehyde used in Example 14 is 2-chloro-1-formyl-3-hydroxymethylenecyclohexene CFHMCH. It was prepared according to the method described by G. A. Reynolds, K. H. Drexhage, J. Org. Chem. 1977, 42, 885. This is a simple and rapid reaction, using DMF, dichloromethane, cyclohexanone and trichlorophosphorus oxide which are all commercial reagents. The synthesis scheme can be summarized as follows:

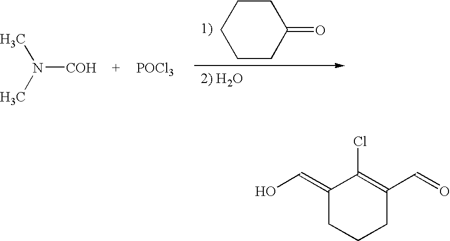

The bisaldehyde CFHMCH exists in the form of an orange-yellow crystalline powder. Its characteristics are the following:

¹H NMR (250 MHz, DMSO-d₆, 25° C.) δ (ppm), J(Hz): 1.58 (q, 2H, CH₂—CH₂—CH₂ J=6); 2.37 (t, 4H, CH₂—CH₂—CH₂ J=6); 10.85 (s, 1H, CHO);

¹³C NMR (62 MHz, DMSO-d₆, 25° C.) δ (ppm): 19.9 CH₂—CH₂—CH₂; 23,6 CH₂—CH₂—CH₂; 146.0 (C—Cl);

Mass (DCI/NH3): [MH⁺]=173, [MNH₄⁺]=190, [MN₂H₇⁺]=207.

### EXAMPLE 1

Preparation of 1,7-diethoxy-1,7-bis(para-methylphenyl)hepta-2,4,6-trienylium tetrafluoroborate (1a):

One equivalent of 1,3,3-triethoxypropene (1.83 g/10.54 mmol) and one equivalent of triethoxymethane (1.75 ml/10.54 mmol) are placed in a 250 ml two-necked round-bottomed flask under argon, at room temperature. Using a dropping funnel, a mixture of two equivalents of acetophenone (2.81 ml/21.08 mmol) and one equivalent of tetrafluoroboric acid at 54% in ether (1.45 ml/10.54 mmol) was added. The solution became gradually violet. After reacting for one hour, about 200 ml of anhydrous ether were added. After stirring for 1 hour, the solution was filtered on sintered material and the precipitate washed with ether, and then dried under vacuum. 2.12 g of salt were obtained in the form of a violet powder whose formula is represented below. The yield is 45%.

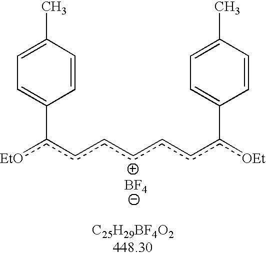

The characteristics of this compound are the following:

¹H NMR (400 MHz, CD₃CN, 25° C.) δ (ppm), J(Hz): 1.49 (t, 6H, J=7.0, CH₃CH₂O); 2.41 (s, 6H, CH₃Ar); 4.46 (q, 4H, J=7.0, CH₃CH₂O); 6.59 (d, J_{H2-H3}=J_{H6-H5}=12.9, 2H, H₂₋₆); 7.03 (t, J_{H4-H3}=J_{H4-H5}=12.9, 1H, H₄); 7.32-7.35 (m, 4H, Hₐᵣₒₘ); 7.49-7.51 (m, 4H, Hₐᵣₒₘ); 7.89 (t, J_{H5-H4}=J_{H3-H4}=J_{H3-H2}=J_{H5-H6}=12.9, 2H, H₃₋₅)

¹³C NMR (100 MHz, CD₃CN, 25° C.) δ (ppm): 14.2 (CH₃CH₂O); 21.4 (CH₃Ar); 70.2 (CH₃CH₂O); 111.3 (C₂₋₆); 130.1 (C₄); 130.3 (Cₐᵣₒₘ); 130.7 (C_{8-8′}); 131.4 (Cₐᵣₒₘ); 145.4 (C_{9-9′}); 169.9 (C₃₋₅); 185.6 (C₁₋₇)

### EXAMPLE 2

Preparation of 1,7-diethoxy-1,7-bis(para-methoxyphenyl)hepta-2,4,6-trienylium tetrafluoroborate (1b):

One equivalent of 1,3,3-triethoxypropene (1.98 g/11.36 mmol) and one equivalent of triethoxymethane (1.89 ml/11.36 mmol) were placed in a 250 ml two-necked round-bottomed flask under argon, at room temperature. Using a dropping funnel, a mixture of two equivalents of 4-methoxy-acetophenone (3.44 g/22.73 mmol), solubilized in 1 ml of anhydrous acetonitrile, and one equivalent of tetrafluoroboric acid at 54% in ether (1.57 ml/11.36 mmol) was added. The solution became gradually violet. After reacting for one hour, about 200 ml of anhydrous ether were added. After stirring for 1 hour, the solution was filtered on sintered material and the precipitate washed with ether, and then dried under vacuum. 2.45 g of salt were obtained in the form of a fine violet powder with a yield of the order of 45% corresponding to the formula represented below.

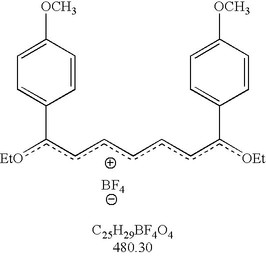

The characteristics of this compound are the following:

¹H NMR (250 MHz, CDCl₃, 25° C.) δ (ppm), J(Hz): 1.53 (t, 6H, J=7, CH₃CH₂O); 3.87 (s, 6H, CH₃O); 4.49 (q, 4H, J=7, CH₃CH₂O); 6.65 (d, J_{H2-H3}=J_{H6-H5}=12.7, 2H, H₂₋₆); 7.01-7.05 (m, 4H, Hₐᵣₒₘ); 7.26 (t, J_{H4-H3}=J_{H4-H5}=12.7, 1H, H₄); 7.55-7.59 (m, 4H, Hₐᵣₒₘ); 7.69 (t, J_{H5-H4=J}_{H3-H4}=J_{H3-H2}=J_{H5-H6}=12.7, 2H, H₃₋₅)

¹³C NMR (63 MHz, CDCl₃, 25° C.) δ (ppm): 14.4 (CH₃CH₂O); 55.9 (CH₃O); 69.2 (CH₃CH₂O); 110.5 (C₂₋₆); 114.8 (Cₐᵣₒₘ); 125.0 (C_{8-8′}); 131.2 (C₄); 132.9 (Cₐᵣₒₘ); 164.41 (C_{9-9′}); 167.0 (C₃₋₅); 183.04 (C₁₋₇).

### EXAMPLE 3

Preparation of 1,7-bis(diethylamino)-1,7-bis(paramethyl-phenyl)-hepta-2,4,6-trienylium tetrafluoroborate (2a):

One equivalent of heptacarbon carboxonium salt 1a (0.448 g/0.1 mmol) was solubilized in about 50 ml of dry acetonitrile in a 100 ml round-bottomed flask under argon, at room temperature. Next, 2.2 equivalents of diethylamine (0.22 ml/2.13 mmol) were added. After stirring overnight, the acetonitrile was evaporated off. The residue was then washed with pentane, and then recrystallized from ethanol. The salt 2a, corresponding to the following formula, was thus isolated in the form of violet crystals, with a yield of 60%.

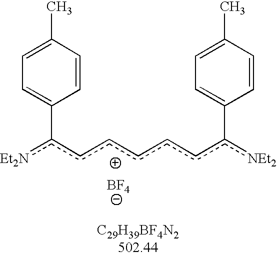

The characteristics of this compound are the following:

¹H NMR (200 MHz, CD₃CN, 25° C.) δ (ppm), J (Hz): 1.17 (m, 12H, (CH₃CH₂)₂N); 2.37 (s, 6H, CH₃Ar); 3.38 (m, 8H, (CH₃CH₂)₂N); 6.09 (m, 2H, H₂₋₆); 6.31 (m, 3H, H₃₋₄₋₅); 7.03-7.06 (m, 4H, Hₐᵣₒₘ); 7.26-7.30 (m, 4H, Hₐᵣₒₘ)

¹H NMR (200 MHz, CD₃CN, 62° C.) δ (ppm), J (Hz): 1.19 (t, 12H, J=7, (CH₃CH₂)₂N); 2.39 (s, 6H, CH₃Ar); 3.41 (q, 8H, J=7, (CH₃CH₂)₂N); 6.08 (m, 2H, H₂₋₆); 6.28 (m, 3H, H₃₋₄₋₅); 7.04-7.09 (m, 4H, Hₐᵣₒₘ); 7.27-7.31 (m, 4H, Hₐᵣₒₘ)

¹³C NMR (63 MHz, CDCl₃, 25° C.) δ (ppm): 21.4 (CH₃Ar); 107.8 (C₂₋₆); 121.6 (C₄); 128.2 (Cₐᵣₒₘ); 129.6 (Cₐᵣₒₘ); 130.0 (C_{8-8′}); 140.2 (C_{9-9′}); 157.8 (C₃₋₅); 167.4 (C₁₋₇)

MS (chemical ionization, NH₃): [M⁺] 415 (100%)

ELEMENTAL ANALYSIS for C₂₉H₃₉BF₄N₂ (M = 502.4 g · mol⁻¹)

| | | | |
|---|---|---|---|
| % theoretical: | C: 69.32 | H: 7.82 | N: 5.58 |
| % experimental: | C: 69.30 | H: 7.78 | N: 5.43 |

VISIBLE-UV: (23° C.)

| | | |
|---|---|---|
| CH₂Cl₂: | λₘₐₓ = 552 nm | ε = 206400 mol⁻¹ · L · cm⁻¹ |

FLUORESCENCE (CH₂Cl₂, T=23° C.) λₑₘᵢₛₛᵢₒₙ=585 nm

IR : (KBr pellet) ν (cm⁻¹): ν_{BF}=1080

Melting point: m.p.=228° C. (decomposition).

### EXAMPLE 4

Preparation of 1,7-bis(diethylamino)-1,7-bis(para-methoxyphenyl)hepta-2,4,6-trienylium tetrafluoroborate (2b):

The procedure described in Example 3 for the preparation of compound 2a was carried out, but using compound 1b obtained in Example 2. The compound corresponding to the following formula was obtained in the form of pink crystals with a yield of 73%.

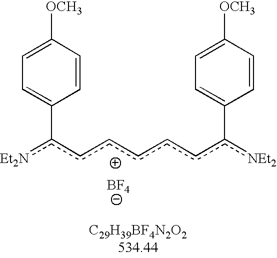

The characteristics of this compound are the following:

¹H NMR (250 MHz, CDCl₃, 25° C.) δ (ppm), J (Hz): 1.26 (m, 12H, (CH₃CH₂)₂N); 3.46(m, 8H, (CH₃CH₂)₂N); 3.86 (s, 6H, CH₃O); 6.01 (m, 2H, H₂₋₆); 6.35 (m, 3H, H₃₋₄₋₅); 6.95-6.99 (m, 4H, Hₐᵣₒₘ); 7.06-7.09 (m, 4H, Hₐᵣₒₘ)

¹³C NMR (63 MHz, CDCl₃, 25° C.) δ (ppm): 55.5 (CH₃O); 108.0 (C₂₋₆); 114.4 (Cₐᵣₒₘ); 121.1 (C₄); 124.9 (C_{8-8′}); 129.9 (Cₐᵣₒₘ); 158.0 (C₃₋₅); 160.8 (C_{9-9′}); 167.4 (C₁₋₇)

MS (chemical ionization, NH₃): [M⁺] 447 (100%)

ELEMENTAL ANALYSIS for C₂₉H₃₉BF₄N₂O₂ (M = 534.4 g · mol⁻¹)

| | | | |
|---|---|---|---|
| % theoretical: | C: 65.17 | H: 7.36 | N: 5.24 |
| % experimental: | C: 65.28 | H: 6.81 | N: 5.05 |

VISIBLE-UV (23° C.):

| | | |
|---|---|---|
| CH₂Cl₂: | λₘₐₓ = 558 nm | ε = 199600 mol⁻¹ · L · cm⁻¹ |
| CHCl₃: | λₘₐₓ = 560 nm | ε = 140700 mol⁻¹ · L · cm⁻¹ |
| CH₃CN: | λₘₐₓ = 550 nm | ε = 201800 mol⁻¹ · L · cm⁻¹ |

FLUORESCENCE (CH₂Cl₂, T=23° C.) λₑₘᵢₛₛᵢₒₙ=593 nm

Melting point: m.p.=204° C. (decomposition).

### EXAMPLE 5

Preparation of 1,7-dimorpholino-1,7-bis(para-methylphenyl)hepta-2,4,6-trienylium tetrafluoroborate (3a):

One equivalent of salt 1a (0.350 g/0.78 mmol) was solubilized in about 50 ml of dry acetonitrile, in a 100 ml round-bottomed flask under argon, at room temperature. Next, 2.2 equivalents of morpholine (0.14 ml/1.59 mmol) were added. After stirring for twelve hours, the acetonitrile was evaporated off. The residue was then washed with pentane, and then recrystallized from ethanol. The salt 3a was thus isolated in the form of violet flakes with green-blue glints, with a 56% yield. It corresponds to the following formula:

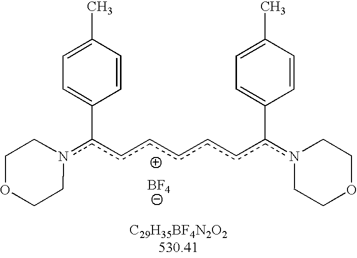

The characteristics of this compound are the following:

¹H NMR (250 MHz, CD₃CN, 25° C.) δ (ppm), J (Hz): 2.38 (s, 6H, CH₃Ar); 3.53 (m, 8H, CH₂N); 3.75 (m, 8H, CH₂O); 6.24-6.29 (m, 2H, H₂₋₆); 6.50-6.53 (m, 3H, H₃₋₄₋₅); 7.06-7.09 (m, 4H, Hₐᵣₒₘ); 7.25-7.28 (m, 4H, Hₐᵣₒₘ)

¹³C NMR (63 MHz, CD₃CN, 25° C.) δ (ppm): 21.5 (CH₃Ph); 50.3 (CH₂N); 66.7 (CH₂O); 109.4 (C₂₋₆); 124.3 (C₄); 129.1 (Cₐᵣₒₘ); 129.5 (C_{8-8′}); 129.9 (Cₐᵣₒₘ); 141.1 (C_{9-9′}); 158.6 (C₃₋₅); 168.0 (C₁₋₇)

MS (chemical ionization, NH₃): [M⁺] 443 (10.2%), 204 (100%)

ELEMENTAL ANALYSIS: for C₂₉H₃₅BF₄N₂O₂ (M = 530.4 g · mol⁻¹):

| | | | |
|---|---|---|---|
| % theoretical: | C: 65.61 | H: 6.65 | N: 5.28 |
| % experimental: | C: 65.63 | H: 5.93 | N: 5.10 |

UV-VISIBLE (23° C.):

| | | |
|---|---|---|
| CH₂Cl₂: | λₘₐₓ = 566 nm | ε = 295600 mol⁻¹ · L · cm⁻¹ |

FLUORESCENCE (CH₂Cl₂, T=23° C.) λₑₘᵢₛₛᵢₒₙ=606 nm

IR (KBr pellet) ν (cm⁻¹):ν_{BF}=1080

Melting point: m.p.=229° C. (decomposition).

### EXAMPLE 6

Preparation of 1,7-dimorpholino-1,7-bis(para-methoxyphenyl)-hepta-2,4,6-trienylium tetrafluoroborate (3b):

The procedure described in Example 5 for the preparation of compound 3a was carried out, but using compound 1b obtained in Example 2. The compound corresponding to the following formula was obtained in the form of a brown powder with a yield of 44%.

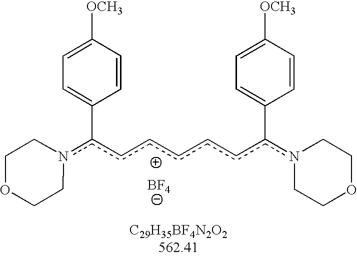

The characteristics of this compound are the following:

¹H NMR (250 MHz, CDCl₃, 25° C.) δ (ppm), J(Hz): 3.55 (m, 8H, CH₂N); 3.66 (m, 8H, CH₂O); 3.85 (s, 6H, CH₃OPh); 6.23-6.25 (m, 2H, H₂₋₆); 6.53-6.57 (m, 3H, H₃₋₄₋₅); 6.96-7.00 (m, 4H, Hₐᵣₒₘ); 7.13-7.17 (m, 4H, Hₐᵣₒₘ)

¹³C NMR (63 MHz, CDCl₃ 25° C.) δ (ppm): 50.4 (CH₂N); 55.6 (CH₃O); 66.7 (CH₂O); 109.6 (C₂₋₆); 113.7-114.7 (Cₐᵣₒₘ); 124.2 (C₄); 124.4 (C_{8-8′}); 130.9 (Cₐᵣₒₘ); 158.6 (C₃₋₅); 161.4 (C_{9-9′}); 167.9 (C₁₋₇)

SM (electronic nebulization): [M⁺] 475.1

ELEMENTAL ANALYSIS: for C₂₉H₃₅BF₄N₂O₄ (M = 562.2 g · mol⁻¹):

| | | | |
|---|---|---|---|
| % theoretical: | C: 61.93 | H: 6.27 | N: 4.98 |
| % experimental: | C: 61.61 | H: 6.22 | N: 4.13 |

VISIBLE-UV (23° C.):

| | | |
|---|---|---|
| CH₂Cl₂: | λₘₐₓ = 569 nm | ε = 146200 mol⁻¹ · L · cm⁻¹ |

FLUORESCENCE (CH₂Cl₂, T=23° C.) λₑₘᵢₛₛᵢₒₙ=613 nm

Melting point: m.p.=209° C. (decomposition).

### EXAMPLE 7

Preparation of 1,7-bis(1-3-dihydrazono)-1,7-bis(para-methylphenyl)hepta-2,4,6-trienylium tetrafluoroborate (4a):

One equivalent (0.64 g/1.42 mmol) of salt 1a was solubilized in about 50 ml of dry acetonitrile, in a 100 ml round-bottomed flask under argon, at room temperature. Two equivalents of hydrazone (0.53 g/2.98 mmol) were then added and an excess of triethylamine (1 ml/7.19 mmol). The reaction was kept stirred overnight. The acetonitrile was then evaporated off. The residue was then washed with pentane, and then dried under vacuum. The solid obtained was recrystallized from acetonitrile The salt 4a was thus isolated in the form of a brown powder with a yield of 36%.

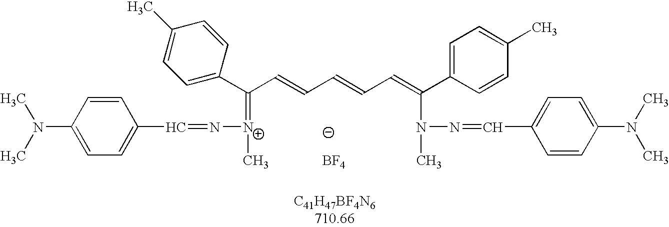

The characteristics of this compound are the following

¹H NMR (250 MHz, CD₃CN, 25° C.) δ (ppm), J(Hz): 2.41 (s, 6H, CH₃Ar); 3.04 (s, 12H, N(CH₃)₂); 3.30 (s, 6H, NCH₃); 6.62 (m, 2H); 6.81 (m, 5H, Hₐᵣₒₘ); 7.00 (m, 2H,); 7.16-7.19 (m, 4H, Hₐᵣₒₘ); 7.31-7.34 (m, 4H, Hₐᵣₒₘ); 7.63-7.67 (m, 4H, Hₐᵣₒₘ); 8.12 (s, 2H, —N═CH—);

MS (chemical ionization): [M⁺] 623 (14%), 477 (100%)

VISIBLE-UV (23° C.):

| | | |
|---|---|---|
| CH₂Cl₂ | λₘₐₓ = 726 nm | ε = 69300 mol⁻¹ · L · cm⁻¹ |

IR (KBr pellet) ν(cm⁻¹): ν_{BF}=1080

Melting point: m.p.=222° C. (decomposition).

### EXAMPLE 8

Preparation of 1,7-bis(1-3-dihydrazono)-1,7-bis(para-methoxyphenyl)hepta-2,4,6-trienylium tetrafluoroborate (4b):

The procedure described in Example 7 for the preparation of compound 4a was carried out, but using compound 1b obtained in Example 2. The compound corresponding to the following formula was obtained in the form of a brown powder with a yield of 21%.

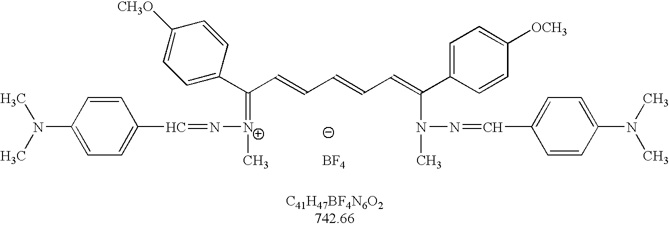

The characteristics of this compound are the following

¹H NMR (250 MHz, CD₃CN, 25° C.) δ (ppm), J(Hz): 3.04 (s, 12H, N(CH₃)₂); 3.33 (s, 6H, NCH₃); 3.85. (s, 6H, CH₃O); 6.66-6.69 (m, 3H, H₂₋₄₋₆); 6.76-6.79 (m, 4H, Hₐᵣₒₘ); 7.03-7.06 (m, 6H, Hₐᵣₒₘ+H₃₋₅); 7.22-7.26 (m, 4H, Hₐᵣₒₘ); 7.67 (m, 4H, Hₐᵣₒₘ); 8.11 (s, 2H, N═CH);

MS (electronic nebulization): [M⁺] 655.5

VISIBLE-UV (23° C.):

| | | |
|---|---|---|
| CH₂Cl₂: | λₘₐₓ = 726 nm | ε = 122000 mol⁻¹ · L · cm⁻¹ |

Melting point: m.p.=225° C. (decomposition).

### EXAMPLE 9

Preparation of 1,7-bis(1-3-dihydrazono)-1,7-bis(para-methylphenyl)hepta-2,4,6-trienylium tetrafluoroborate (5a):

One equivalent (426.6 mg/0.951 mmol) of salt 1a was solubilized in about 50 ml of dry acetonitrile, in a 100 ml round-bottomed flask under argon, at room temperature. 2.1 equivalents of hydrazone (328 mg/2 mmol) were then added and an excess of triethylamine (0.6 ml/5 mmol). The reaction was kept stirred overnight. The acetonitrile was then evaporated off. The residue was washed with pentane and then dried under vacuum. The solid obtained was recrystallized from ethanol. The salt 5a was thus isolated in the form of a blue-green powder with a yield of 0.12%.

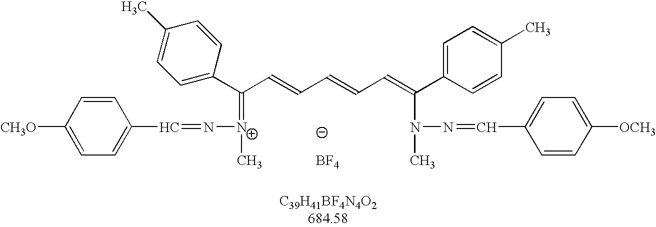

The characteristics of this compound are the following

¹H NMR (400 MHz, CDCl₃, 25° C.) δ (ppm), J(Hz): 2.37 (s, 6H, CH₃Ar); 3.35 (s, 6H, CH₃N); 3.83 (s, 6H, CH₃O); 6.60-6.62 (m, 3H, H₂₋₄₋₆); 6.90-6.95 (m, 6H, Hₐᵣₒₘ+H₃₋₅); 7.12-7.14 (m, 4H, Hₐᵣₒₘ), 7.26-7.28 (m, 4H, Hₐᵣₒₘ); 7.73-7.75 (m, 4H, Hₐᵣₒₘ); 8.20 (s, 2H, —N═CH);

MS (FAB>0, MNBA): [M⁺] 597 (85.5%), 327 (100%)

ELEMENTAL ANALYSIS for C₃₉H₄₁BF₄N₄O₂ · 0.5 H₂O
(M = 684.3 g · mol⁻¹)

| | | | |
|---|---|---|---|
| % theoretical: | C: 67.54 | H: 6.10 | N: 8.08 |
| % experimental: | C: 67.74 | H: 5.88 | N: 8.16 |

VISIBLE-UV (23° C.):

| | | |
|---|---|---|
| CH₂Cl₂: | λₘₐₓ = 679 nm | ε = 136700 mol⁻¹ · L · cm⁻¹ |

FLUORESCENCE (CH₂Cl₂, T=23° C.) λₑₘᵢₛₛᵢₒₙ=720 nm m.p=222° C.

### EXAMPLE 10

Preparation of 1-ethoxy-7-diethylamino-1,7-bis(para-methylphenyl)hepta-2,4,6-trienylium tetrafluoroborate (6a):

One equivalent (0.448 g/1.08 mmol) of salt 1a was solubilized in about 50 ml of dry acetonitrile, in a 100 ml round-bottomed flask under argon, at room temperature. One equivalent of diethylamine (0.11 ml/1.08 mmol) was then added. After reacting for twelve hours, the acetonitrile was evaporated off. The residue was washed with pentane and then dried under vacuum. The salt 6a corresponding to the following formula was isolated in the form of an orange-red powder.

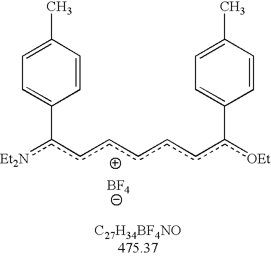

The characteristics of this compound are the following:

¹H NMR (200 MHz, CD₃CN, 25° C.) δ (ppm), J(Hz) 1.12-1.46 (m, 9H, (CH₃CH₂)₂N+CH₃CH₂O); 2.35-2.47 (m, 6H, CH₃Ar); 3.06-4.11 (m, 6H, (CH₃CH₂)₂N+CH₃CH₂O); 5.92→7.44 (m, 8+5=13H, H₃₋₅; H₂₋₄₋₆, Hₐᵣₒₘ)

MS: [M⁺] 388

EXAMPLE 11

Preparation of 1-ethoxy-7-diethylamino-1,7-bis(para-methoxyphenyl)hepta-2,4,6-trienylium tetrafluoroborate (6b):

The procedure described in Example 10 for the preparation of compound 6a was carried out, but using compound 1b obtained in Example 2. The compound corresponding to the following formula was obtained in the form of a bright black powder with a yield of 92%.

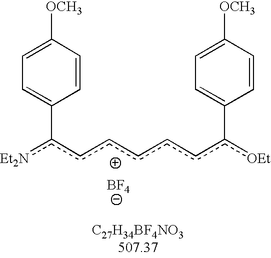

The characteristics of this compound are the following:

¹H NMR (250 MHz, CDCl₃, 25° C.) δ (ppm), J(Hz): 1.23-1.41 (m, 9H, (CH₃CH₂)₂N+CH₃CH₂O); 3.12-4.07 (m, 12H, (CH₃CH₂)₂N+CH₃CH₂O+CH₃OAr); 5.85→7.45 (m, 12H, H₃₋₅; H₂₋₄₋₆, Hₐᵣₒₘ)

MS : [M⁺] 420

### EXAMPLE 12

Preparation of 1-ethoxy-7-hydrazono-1,7-bis(para-methylphenyl)hepta-2,4,6-trienylium tetrafluoroborate (7a):

0.67 g (1.48 mmol) of salt 1a was solubilized in about 50 ml of dry acetonitrile, in a 100 ml round-bottomed flask under argon, at room temperature. 0.26 g (1.48 mmol) of hydrazone was then added. The reaction was kept stirred overnight. The acetonitrile was then evaporated off. The residue was then washed with pentane and then dried under vacuum. The solid obtained was recrystallized from ethanol. The salt 7a corresponding to the following formula was then isolated in the form of dark green crystals with a yield of 37%.

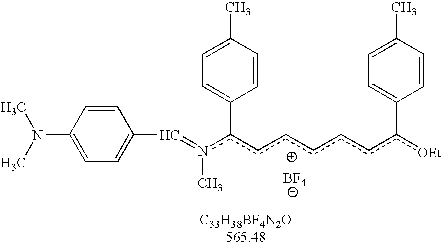

The characteristics of this compound are the following

¹H NMR (250 MHz, CD₃CN, 25° C.) δ (ppm), J(Hz): 1.22-1.40 (m, 3H, (CH₃CH₂)N); 2.37-2.50 (m, 6H, CH₃Ar); 3.00-3.16 (m, 6H, N(CH₃)₂); 3.49-3.56 (m, 3H, NCH₃); 3.83-4.10(m, 2H, CH₃CH₂O); 5.87→7.90 (m, 17H, Hₐᵣₒₘ+H₂₋₃₋₄₋₅₋₆);

MS: [M⁺] 492

VISIBLE-UV (23° C.):

| | | |
|---|---|---|
| CH₂Cl₂ | λₘₐₓ = 577 nm | ε = 41500 mol⁻¹ · L · cm⁻¹ |

IR (KBr pellet) ν (cm⁻¹): ν_{BF}=1080

Melting point: m.p.=146° C. (decomposition).

### EXAMPLE 13

Preparation of 1-hydrazono-7-diethylamino-1,7-bis(para-methylphenyl)hepta-2,4,6-trienylium tetrafluoroborate (8a):

0.223 g (0.39 mmol) of hemicarboxonium salt 7a was solubilized in 15 ml of dry acetonitrile, in a 100 ml round-bottomed flask under argon, at room temperature. 0.04 ml (0.39 mmol) of diethylamine was then added. After stirring overnight, the acetonitrile was evaporated off. The residue obtained was then recrystallized from absolute ethanol. The salt 8a corresponding to the following formula was thus isolated in the form of a dark blue crystalline powder with a yield of 37%.

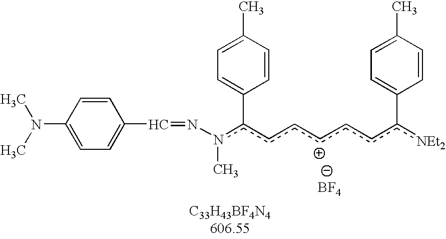

The characteristics of this compound are the following:

¹H NMR (250 MHz, CD₃CN, 25° C.) δ (ppm), J(Hz) 1.25 (m, 6H, (CH₃CH₂)₂N); 2.38 (s, 6H, CH₃Ar); 3.01 (s, 6H, (CH₃)₂N); 3.20 (s, 3H, CH₃—N); 3.54 (m, 4H, (CH₃CH₂)₂N); 6.33-6.47 (m, 4H, Hₐᵣₒₘ); 6.73-6.76 (m, 3H, H₂₋₄₋₆); 7.07-7.14 (m, 4H, Hₐᵣₒₘ); 7.27-7.32 (m, 4H, Hₐᵣₒₘ); 7.57-7.60 (m, 2H, H₃₋₅); 7.97 (s, 1H, H₁₀)

3C NMR (62 MHz, CD₃CN, 25° C.) δ (ppm): 21.5 (CH₃Ar); 37.0 (CH₃N); 40.5 ((CH₃)₂N); 109.9 (C₆ or C₂); 111.8 (C₆ or C₂); 112.9 (C_{arom hydra}); 122.7 (C11); 123.7 (C₄); 130.4-130.5 (Cₐᵣₒₘ); 131.7 (C_{8-8′}); 141.6-141.2 (C_{9-9′}); 147.7 (C3 or C5); 153.4 (C₁₂); 156.3 (C₃ or C₅); 160.1 (C₁₀); 163.1(C₁ or C₇); 171.6 (C₁ or C₇)

MS : [M⁺] 519 (15%); [(Me)₂N—C₆H₄—CH═NH₂+] 149 (100%)

IR (KBr pellet) ν (cm⁻¹): ν_{BF} = 1080
VISIBLE-UV (23° C.):

| | | |
|---|---|---|
| CH₂Cl₂ | λₘₐₓ = 639 nm | ε = 97000 mol⁻¹ · L · cm⁻¹ |

Melting point: m.p.=215° C. (decomposition).

### EXAMPLE 14

Synthesis of a Nonacarbon 1,9-diarylcarboxonium Compound (9a)

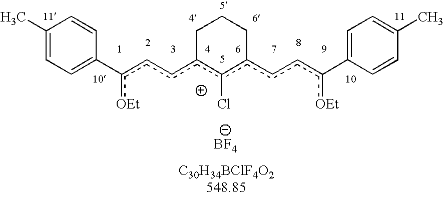

One equivalent of bisaldehyde (1.235 g/7.15 mmol), 4 equivalents of triethoxymethane (4.75 ml/28.6 mmol), and about 4 ml of anhydrous diethyl ether are introduced into a 250 ml two-necked round-bottomed flask. The practically homogeneous mixture is magnetically stirred under an argon stream. A mixture of two equivalents of acetophenone (1.91 ml/14.3 mmol) and one equivalent of tetrafluoroboric acid at 54% in ether (0.98 ml/7.15 mmol) is then added dropwise. The color of the reaction medium changes as the addition progresses, passing from red to blue, and then to green. After a few minutes the reaction medium collects into a mass, and about 200 ml of anhydrous diethyl ether are then added. The reaction medium is then kept stirred for 5 to 10 minutes, and then filtered under argon on a No. 3 sintered material. The violet red precipitate with metallic glints is washed with 200 ml of anhydrous diethyl ether. 2.80 g of a powder are obtained after drying under vacuum of which the composition, determined by proton NMR, corresponding to a mixture consisting of 86 mol % of the expected compound (9a), and 14 mol % of the diketone compound (9a′) corresponding to the following formula:

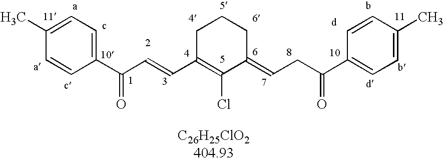

The characteristics of the compound (9a) are the following:

¹H NMR (250 MHz, CDCl₃, 25° C.) δ (ppm), J(Hz): 1.54 (t, 6H, J=6.9, CH₃CH₂O); 1.96 (quint, 2H, J=5.7, CH_{2(5′)}); 2.43 (s, 6H, CH₃Ar); 2.76 (t, 4H, J=5.7, CH_{2(4′-6′)}); 4.50 (q, 4H, J=6.9, CH₃CH₂O) i 6.52 (d, 2H, J=13, CH₍₂₋₈₎); 7.30 7.48 (syst. AB, 8H, J=8.2, CHₐᵣₒᵣₘ); 8.24 (d, 2H, J=13.0, CH₍₃₋₇₎);

¹³C NMR (63 MHz, CDCl₃, 25° C.) δ (ppm), J(Hz):

14.4 (CH₃CH₂O); 20.4 (CH_{2(5′)}); 21.8 (CH₃Ar); 26.9 (CH_{2(4′-5′)}); 69.3 (CH₃CH₂O); 108.0 (C₍₂₋₈₎); 129.6 (CHₐᵣₒₘ); 130.6 (C₄₋₆); 130.8 (CHₐᵣₒₘ); 133.6 (C_{10-10′}); 144.6 (C_{(11-11′)}); 156.4 (C₍₃₋₇₎); 160.1 (C₅); 182.6 (C₍₁₋₉₎);

The characteristics of the diketone compound (9a′) are the following:

¹H NMR (400 MHz, CDCl₃, 25° C.) δ (ppm), J(Hz): 1.82 (quint, 2H, J=6.2, CH_{2(5′)}); 2.39 (s, 6H, CH₃Ar) 2.52 (quint, 4H, J=6.2, CH_{2(4′-6′)}); 3.85 (d, 2H, J=7.2, CH₂₍₈₎); 6.63 (t, 1H, J=7.2), CH₍₇₎; 7.00 (d, 1H, J=15.6, CH₍₂₎); 7.25 and 7.83 (syst. AB, 8H, J=8.2, CHₐᵣₒₘ); 8.18 (d, 1H, J=15.6, CH₍₃₎);

¹³C NMR (100 MHz, CDCl₃, 25° C.) δ (ppm):

21.4 (C_{5′}); 21.8 (CH₃Ar); 27.3 and 27.4 (C_{4′-6′)}; 38.7 (C₈); 124.0 (C₂); 124.8 (C₇); 128.6 and 128.8, and 129.4 and 129.6 (CHₐᵣₒₘ); 131.8 (C₄); 134.2 (C_{(10-10′)}); 135.8 (C₆); 138.2 (C₅—Cl); 142.2 (C₃); 143.6 and 144.4 (C_{(11-11′)) ;} 190.5 (C₁); 196.5 (C₉);

MS (chemical ionization, NH₃) [MH⁺]=405

VISIBLE-UV: (23° C.):

| | | |
|---|---|---|
| CH₂Cl₂: | λₘₐₓ = 349 nm | ε = 24000 mol⁻¹ · L · cm⁻¹ |

FLUORESCENCE (CH₂Cl₂, T=23° C.) λₑₘᵢₛₛᵢₒₙ=396 nm IR (KBr) v (cm⁻¹) 1651 and 1680 (C═O)

### EXAMPLE 15

Synthesis of the Cyanine (10a)

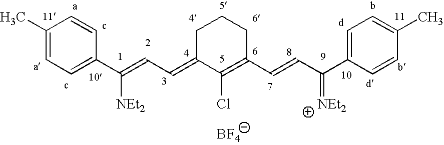

399.6 mg of the product obtained according to the method of Example 14, that is to say 0.728 mmol of carboxonium salt (9a), were dissolved in about 50 ml of dry acetonitrile under argon, at room temperature, in a 100 ml round-bottomed flask. Next, 0.150 ml, that is to say 1.45 mmol, of diethylamine was added, the proportion of the reagents thus being 1 equivalent of compound (9a) per two equivalents of diethylamine. After stirring overnight, the solution was filtered on No. 3 sintered material in order to separate the cyanine (10a) from the diketone (9a′) introduced into the reaction medium at the same time as the compound (9a) which precipitates. The acetonitrile of the filtrate was then evaporated off and the residue washed with ether, and then recrystallized from ethanol. The cyanine (10a) (0.263 g) was thus isolated in the form of green flakes, which corresponds to a yield of 82%.

The characteristics of (10a) are given below

¹H NMR (400 MHz, CDCl₃, 25° C.) δ (ppm), J(Hz):

1.19 (m, 12H, ((CH₃—CH₂)₂N); 1.78 (quint, 2H, J=6.0, CH_{2(5′)}); 2.35 (s, 6H, CH₃—Ar); 2.46 (t, 4H, J=6.0, CH_{2(4′-6′)}); 3.46 (m, 8H, ((CH₃—CH₂)₂N); 5.98 (d, 2H, J=13.2, H₂₋₈); 7.03 (part A of a syst. AB, 4H, J=8.2, CHₐᵣₒₘ); 7,05 (d, 2H, 3J=13.2, CH₍₃₋₇₎); 7.21 (part B of a syst. AB, 4H, J=8.2, CHₐᵣₒₘ);

NMR¹³C (100 MHz, CDCl₃, 25° C.) δ (ppm): 13.4 (CH₃—CH₂N); 21.1 (CH_{2(5′)}); 21.6 (CH₃—Ar); 27.0 (CH_{2(4′-6′)}); 47.7 (CH₃—CH₂N); 106.0 (CH₍₂₋₈₎); 124.4 (C₍₄₋₆₎); 128.5 (CH_{(cc′-dd′)}); 129.6 (CH_{(aa′-bb′)}); 130.6 (C_{(10-10′)}); 140.3 (C_{(11-11′)}); 150.0 (CH₍₃₋₇₎); 150.7 (C₅); 167.8 (C₍₁₋₉₎).

MS (electronic nebulization) [M⁺] 515

ELEMENTAL ANALYSIS for C₃₄H₄₄BClF₄N₂ (M = 602.99 g · mol⁻¹)

| | | | |
|---|---|---|---|
| % theoretical: | C: 67.72 | H: 7.35 | N: 4.65 |
| % experimental: | C: 67.84 | H: 7.15 | N: 4.55 |

Visible-UV: (23° C.)

| | | |
|---|---|---|
| CH₂Cl₂: | λₘₐₓ = 695 nm | ε = 240000 mol⁻¹· 1 · cm⁻¹ |

FLUORESCENCE (CH₂Cl₂, T=23° C.)

λ_{exc}=686 nm/λₑₘᵢₛₛᵢₒₙ=719 nm

IR: (KBr pellet) ν (cm⁻¹): ν_{BF}=1080

Melting point m.p.=235-237° C. (decomposition)

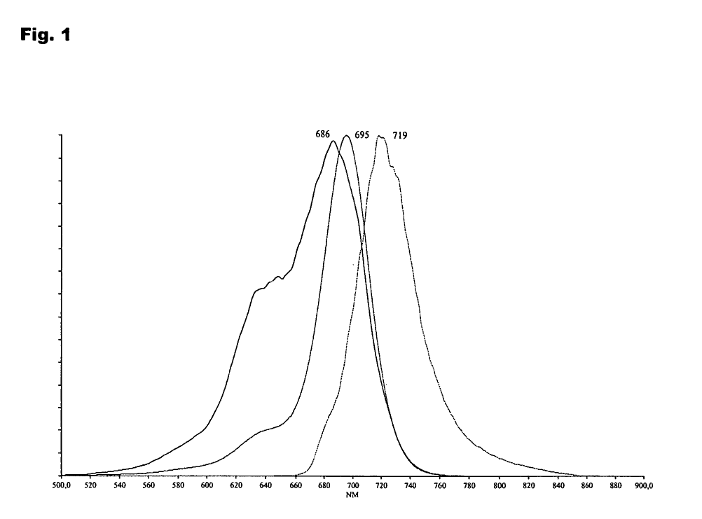

FIG. 1 represents the UV spectrum (λₘₐₓ=695 nm), the emission spectrum (λ=719 nm), and the excitation spectrum (λ=686 nm), in dichloromethane.

### EXAMPLE 16

Synthesis of the Cyanine (11a)

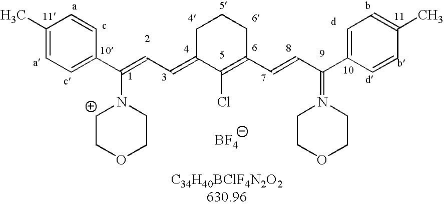

340.5 mg of the product obtained according to the method of Example 14, that is to say 0.620 mmol of carboxonium salt (9a), were dissolved in about 50 ml of dry acetonitrile, in a 100 ml round-bottomed flask under argon, at room temperature. Next, 0.11 ml, that is to say 1.24 mmol, of morpholine was added, the proportion of the reagents thus being one equivalent of compound (9a) per two equivalents of morpholine. The reaction medium was kept stirred overnight, and then the solution was filtered on No. 3 sintered material in order to separate the salt (lla) from the diketone (9a′). The filtrate was evaporated and the residue washed with ether, and then recrystallized from ethanol. The salt (11a), corresponding to the preceding formula, was thus isolated in the form of a fine green crystalline powder with a yield of 30%.

The characteristics of (11a) are given below:

NMR¹H (400 MHz, CDCl₃, 25° C.) δ (ppm), J(Hz): 1.79 (quint, 2H, J=6.0, CH_{2(5′)}); 2.36 (s, 6H, CH₃—Ar); 2.53 (m, 4H, J=6.0, CH_{2(4′-6′)}); 3.54 (t, 8H, J=4.5, CH₂N), 3.78 (t, 8H, J=4.5, CH₂O), 6.23 (d, 2H, J=13.2, CH₍₃₋₇₎); 7.13 (part A of a syst. AB, 4H, J=7.8, CHₐᵣₒₘ); 7.21 (d, 2H, J=13.2, CH₂₋₈); 7.23 (part B of a syst. AB, 4H, 3J=7.8, CHₐᵣₒₘ);

¹³C NMR (100 MHz, CDCl₃, 25° C.) δ (ppm) 21.1 (CH_{2(5′)}); 21.6 (CH₃—Ar); 26.9 (CH_{2(4′-6′)}); 50.9 (CH₂N); 66.8 (CH₂O); 108.0 (CH₍₂₋₈₎); 127.5 (C₍₄₋₆₎); 129.5 (CH_{(cc′-dd′)}); 129.8 (CH_{(aa′-bb′)}); 130.1 (C_{(10-10′)}); 141.2 (C_{(11-11′)}); 149.5 (CH₍₃₋₇₎); 150.1 (C₅); 168.1 (C₍₁₋₉₎);

MS (electronic nebulization): [M+]=543

ELEMENTAL ANALYSIS for C₃₄H₄₀BClF₄N₂O₂·
0.5 H₂O (M = 630.96 g · mol⁻¹)

| | | | |
|---|---|---|---|
| % theoretical: | C: 63.81 | H: 6.46 | N: 4.36 |
| % experimental: | C: 63.63 | H: 6.00 | N: 4.25 |

Visible-UV: (23° C.)

| | | |
|---|---|---|
| CH₂Cl₂: | λₘₐₓ = 706 nm | ε = 186000 mol⁻¹· 1 · cm⁻¹ |

FLUORESCENCE (CH₂Cl₂, T=23° C.)

λ_{exc}=649 nm/λₑₘᵢₛₛᵢₒₙ=743 nm

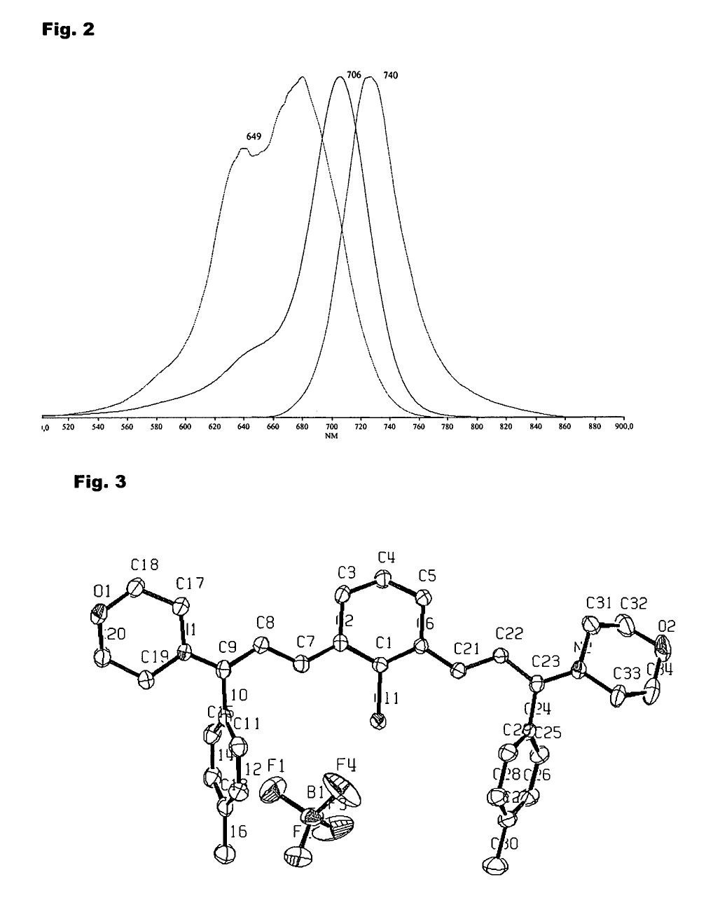

FIG. 2 represents the UV spectrum (λₘₐₓ=706 nm), the emission spectrum (λ=740 nm), and the excitation spectrum (λ=649 nm), in dichloromethane.

IR : (KBr pellet) ν (cm⁻¹) ν_{BF}=1080

Melting point: m.p.=255-257° C. (decomposition)

FIG. 3 gives the structure of compound (11a) as determined by X-rays.

### EXAMPLE 17

Synthesis of the Cyanine (12a)

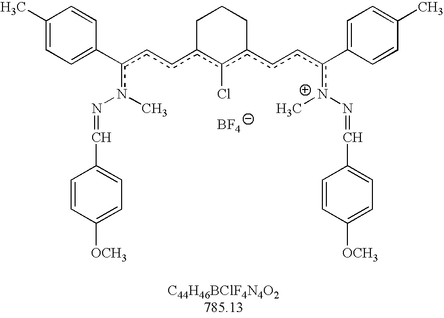

368.8 mg of the product obtained according to the method of Example 14, that is to say 0.513 mmol of salt (9a) were dissolved in about 50 ml of dry acetonitrile, in a 100 ml round-bottomed flask under argon, at room temperature. 168 mg, that is to say 1.26 mmol, of p-methoxyphenyldimethyl-hydrazone and some triethylamine (10 μl) were then added. The proportion of the reagents is thus 1 equivalent of compound (9a) per two equivalents of hydrazone. The reaction was kept stirred overnight. The solution is then filtered on a No. 3 sintered material in order to separate the salt (12a) from the diketone (9a′) which precipitates. The filtrate was then evaporated, and then the residue washed with ether and dried under vacuum. The solid obtained was recrystallized from acetonitrile. The salt (12a) was thus isolated in the form of a fine green powder with a yield of 10%.

The characteristics of (12a) are the following

¹H NMR (250 MHz, DMSO-d₆, 25° C.) δ (ppm), J(Hz): 1.86 (m, 2H, CH_{2(5′)}); 2.40 (s, CH₃Ar); 2.63 (m, CH_{2(4′-6′)}); 3.42 (s, 6H, NCH₃); 3.81 (s, 6H, OCH₃); 6.96-7.09 (m, 6H, CHₐᵣₒₘ and CH₂₋₈); 7.22-7.30 (m, 10H, CHₐᵣₒₘ and CH₃₋₇); 7.70 (m, 4H, CHₐᵣₒₘ); 8.30 (s, 2H, CHN);

¹³C NMR (100 MHz, CDCl₃, 25° C.) 8 (ppm) 20.5 (CH₂ (5′)); 21.0 (CH₃—Ar); 26.3 (CH_{2(4′-6′)}); 37.5 (CH₃N); 55.1 (CH₃O); 110.2 (CH₂₋₈); 114.3 (CHₐᵣₒₘ); 126.0 (C_{quat}, C₄₋₆); 127.4 (C_{quat}, C—C₁₋₉); 129.0 (CHₐᵣₒₘ); 129.3 (CHₐᵣₒₘ); 129.4 (C_{quat}, C—OCH₃); 129.7 (CHₐᵣₒₘ); 140.3 (C_{quat}, C—CH₃); 147.5 (CH₃₋₇); 147.9 (CHN); 148.8 (C_{quat}, C₅); 161.7 (C_{quat}, C—CHN); 163.8 (C_{quat}, C₁₋₉);

MS (electronic nebulization) [M+]=697

ELEMENTAL ANALYSIS for C₄₄H₄₆BClF₄N₄O₂ · 0.5 H₂O
M = 784.333 g · mol⁻¹

| | | | |
|---|---|---|---|
| % theoretical: | C: 66.55 | H: 5.97 | N: 7.05 |
| % experimental: | C: 65.99 | H: 5.82 | N: 6.85 |

Visible-UV: (23° C.)

| | | |
|---|---|---|
| CH₂Cl₂: | λₘₐₓ = 822 nm | ε = 210000 mol⁻¹· 1 · cm⁻¹ |

FLUORESCENCE (CH₂Cl₂, T=23° C.) λ_{exc}=827 nm/λₑₘᵢₛₛᵢₒₙ=843 nm

### EXAMPLE 18

Synthesis of the Cyanine (13a)

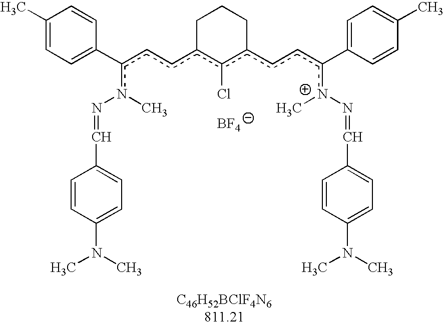

551.9 mg of the product obtained according to the method of Example 14, that is to say 0.767 mmol of salt (9a), were dissolved in about 50 ml of dry acetonitrile, in a 100 ml round-bottomed flask under argon, at room temperature. 271.8 mg, that is to say 1.534 mmol, p-dimethylaminophenyl-dimethylhydrazone and some triethylamine (10 μl) were then added. The proportion of the reagents is thus 1 equivalent of compound (9a) per two equivalents of hydrazone. The reaction was kept stirred overnight. The solution was then filtered on No. 3 sintered material in order to separate the salt (13a) from the diketone (9a′) which precipitates. The filtrate was evaporated, and the residue washed with ether and dried under vacuum. The solid obtained was recrystallized from acetonitrile. The salt (13a) was thus isolated in the form of a fine red powder with a yield of 15%.

The characteristics of (13a) are the following:

¹H NMR (250 MHz, DMSO-d₆, 25° C.) δ (ppm), J(Hz): 1.82 (m, 2H, CH_{2(5′)}); 2.41 (CH₃Ar); 2.62 (CH_{(4′-5′)}); 3.01 (N(CH₃)₂); 3.43 (CH₃N); 6.79 (part A of a syst. AB, 4H, J=8.06, CHₐᵣₒₘ); 7.05 (d, 2H, J=13.0, CH₂₋₈); 7.15 (d, 2H, J=13.0, CH₃₋₇); 7.29 and 7.38 (syst. YZ, 8H, J=7.52, CHₐᵣₒₘ); 7.64 (part B of a syst. AB, 4H, J=8.06, CHₐᵣₒₘ); 8.30 (s, 2H, CHN);

¹³C NMR (100 MHz, CDCl₃, 25° C.) δ (ppm): 20.6 (CH_{2(5′))}; 21.0 (CH₃—Ar); 26.3 (CH_{2(4′-6′)}); 37 7 (CH₃N); 39.6 ((CH₃)₂N); 109.8, 111.8, 120.6 (C_{quat}); 126.4 (C_{quat}); 129.1, 129.5, 129.7, 129.8 (C_{quat}); 140.0 (C_{quat}); 145.7, 146.6 (C_{quat}); 149.0, 152.2 (C_{quat}); 162.5 (C_{quat});

MS (electronic nebulization): Electrospray [M+]=723

ELEMENTAL ANALYSIS for C₄₆H₅₂BClF₄N₆ · 0.5 H₂O
(M = 810.397 g · mol⁻¹)

| | | | |
|---|---|---|---|
| % theoretical: | C: 67.36 | H: 6.51 | N: 10.25 |
| % experimental: | C: 67.15 | H: 6.30 | N: 10.15 |

Visible-UV: (23° C.)

| | | |
|---|---|---|
| CH₂Cl₂: | λₘₐₓ = 863 nm | ε = 160000 mol⁻¹· 1 · cm⁻¹ |

FLUORESCENCE (CH₂Cl₂, T=23° C.) λₑₘᵢₛₛᵢₒₙ=no fluorescence

1. A compound corresponding to formula (I) 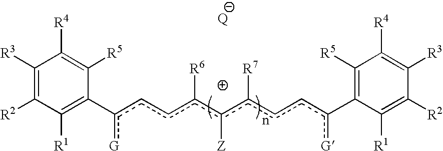
in which:
Q is an anion of a strong acid;
G and G′ represent, independently of each other, an OEt group, an amino group, a phosphaimino group, an amidino group, a guanidino group, a hydrazino group, a hydrazono group, or a multivalent radical linked at at least one of its other ends to a radical corresponding to formula (I′) 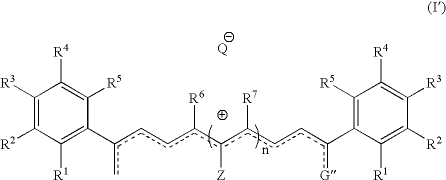
in which G″ represents an OEt group, an amino group, a phosphaimino group, an amidino group, a guanidino group, a hydrazino group, a hydrazono group, or a multivalent radical;
R¹ to R⁵ represent, independently of each other, a hydrogen, a halogen, an alkyl radical, an alkyloxy radical having from 1 to 15 carbon atoms or an acetamido group CH₃C(O)HN—;
Z represents H or a halogen,
n is 0 or 1;
R⁶ and R⁷ represent, independently of each other, H, or alternatively R⁶ and R⁷ form together a 3- or 4-membered biradical optionally carrying one or more substituents chosen from methyl or ester groups, it being understood that R⁶ represents H when n=0.

2. The compound as claimed in claim 1, characterized in that Q is chosen from BF₄⁻, CF₃SO₃⁻, ClO₄⁻, I⁻, Br⁻ and Cl⁻.

3. The compound as claimed in claim 1, characterized in that it corresponds to formula (II)

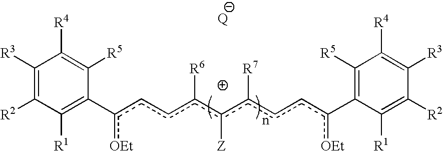

4. The compound as claimed in claim 1, characterized in that it corresponds to formula (III) 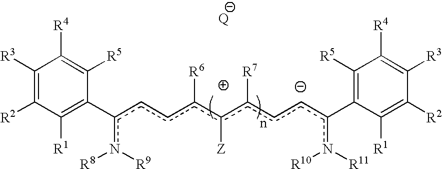
in which
R⁸, R⁹, R¹⁰ and R¹¹ are chosen, independently of each other, from:
H;
alkyl radicals having from 1 to 12 carbon atoms;
phenyl radicals optionally carrying substituents chosen, independently of each other, from H, halogens, alkyl or alkyloxy radicals having from 1 to 15 carbon atoms or the acetamido group CH₃C(O)HN;
the groups —N═CHA and —NHA in which A represents a phenyl group optionally carrying one or more alkyloxy or dialkylamino substituents, it being understood that when R⁸ (respectively R¹⁰) is —N═CHA and —NHA, R⁹ (respectively R¹¹) is a methyl group.
or alternatively R⁸ and R⁹ and/or R¹⁰ and R¹¹ form together an aliphatic ring optionally comprising an oxygen atom.

5. The compound as claimed in claim 1, characterized in that it corresponds to formula (IV) 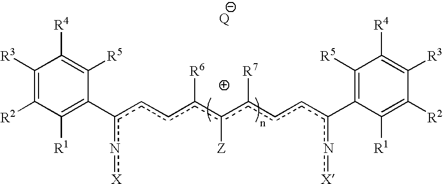
in which X and X′ represent, independently of each other, R″₃P, R″₂N(R′)C, (R″₂N)₂C or NR″₂, R″ representing an alkyl or a phenyl.

6. The compound as claimed in claim 5, characterized in that R″ is an alkyl having from 1 to 4 carbon atoms.

7. The compound as claimed in claim 1, characterized in that it corresponds to formula (V) 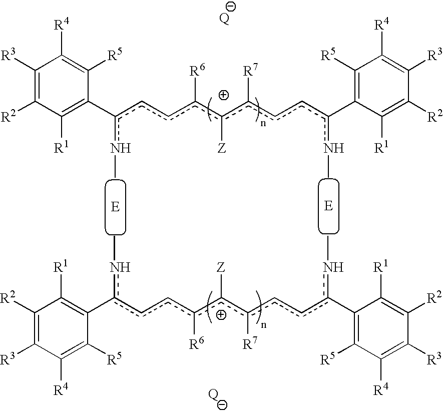
in which E is a group —(CH₂)ₙ— with n=3 to 9, or —(CH₂)₂O(CH₂)₂O(CH₂)₂—.

8. The compound as claimed in claim 1, characterized in that it corresponds to formula (VI) 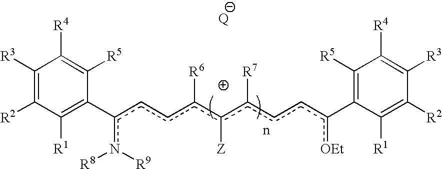
in which
R⁸ and R⁹ are chosen, independently of each other, from:
H;
alkyl radicals having from 1 to 12 carbon atoms;
phenyl radicals optionally carrying substituents chosen, independently of each other, from H, halogens, alkyl or alkyloxy radicals having from 1 to 15 carbon atoms or the acetamido group CH₃C(O)HN;
the groups —N═CHA and —NHA in which A represents a phenyl group optionally carrying one or more alkyloxy or dialkylamine substituents, it being understood that when R⁸ is —N═CHA and —NHA, R⁹ is a methyl group.
or alternatively R⁸ and R⁹ form together an aliphatic ring optionally comprising an oxygen atom.

9. The compound as claimed in claim 1, characterized in that it corresponds to formula (VII) 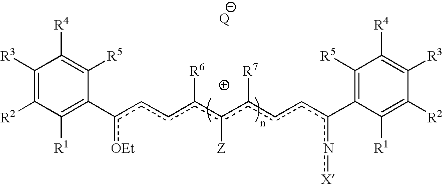
in which X′ represents R″₃P, R″₂N(R′)C, (R″₂N)₂C or NR″₂, R″ representing an alkyle or a phenyl.

10. The compound as claimed in claim 1, characterized in that one of the substituents G or G′ is a multivalent group linked at each of its ends to a group corresponding to formula (I′)

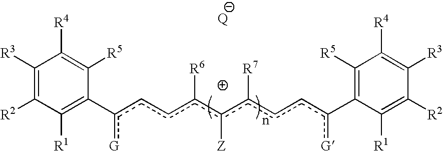

11. The compound as claimed in claim 1, in which n is 0 and R⁶ is H, corresponding to formula

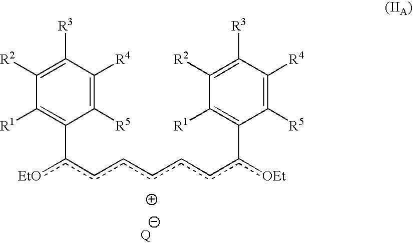

12. The compound as claimed in claim 1, in which n is 1, corresponding to formula

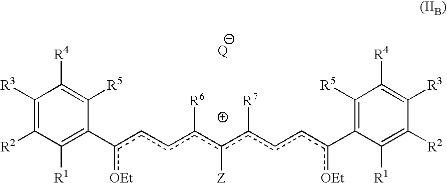

13. A method for preparing a compound (II_{A}) as claimed in claim 11, characterized in that it consists in reacting an aryl ketone AK with a mixture of triethoxymethane (TEM) and 1,3,3-triethoxypropene (TEP) in the presence of a strong acid chosen from HBF₄, CF₃SO₃H, HClO₄, HI, HBr or HCl, under an inert atmosphere, in an anhydrous medium, by using quantities of reagents such that the mol ratios are such that 0.25≦TEP/TME≦3 and 1/4≦AK/TEM+TEP≦2, the aryl ketone corresponding to formula Ar—C(O)R′ in which Ar represents a phenyl radical carrying the substituents R¹ to R⁵ and R′ represents an alkyl radical having from 1 to 5 carbon atoms.

14. The method as claimed in claim 13, characterized in that the temperature of the reaction medium is between −5° C. and 80° C.

15. The method as claimed in claim 13, characterized in that the TEP/TME ratio is equal to 1 and the AC/TEM+TEP ratio is equal to 1/2.

16. The method as claimed in claim 13, characterized in that compound (I) is recovered by precipitation, filtration, washing and drying.

17. A method for preparing a compound (II_{B}) as claimed in claim 12, characterized in that it consists in reacting an aryl ketone ArC(O)R′ in which Ar represents a phenyl radical carrying the substituents R¹ to R⁵ and R′ represents an alkyl radical having from 1 to 5 carbon atoms with a mixture of triethoxymethane (TEM) and a bisaldehyde (BA) in the presence of a strong acid, under an inert atmosphere and in an anhydrous medium, the quantities of reagents being such that the mol ratios are the following: 1/6≦BA/TEM<1/3 and 2/7≦AK/TEM+BA<0.5.

18. The method as claimed in claim 17, characterized in that the reaction is carried out at a temperature between −5° C. and 80° C.

19. The method as claimed in claim 17, characterized in that the BA/TEM ratio is equal to 1/4 and the AK/TEM+BA ratio is equal to 2/5.

20. The method as claimed in claim 17, characterized in that the bisaldehyde corresponds to formula

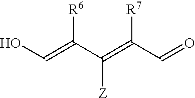

21. The method as claimed in claim 20, characaterized in that the bisaldehyde is chosen from 2-chloro-1-formyl-3hydroxymethylenecyclohexene, 1-formyl-3-hydroxymethylenecyclohexene, 2-chloro-1-formyl-3-hydroxymethylenecyclopentene, 1-formyl-3-hydroxymethylenecyclopentene, and a glutaconaldehyde salt.

22. The method as claimed in either of claims 11 and 12, characterized in that the strong acid is chosen from HBF₄, CF₃SO₃H, HClO₄, HI, HBr or HCl.

23. A method for preparing a symmetrical streptocyanine (III), characterized in that it consists in reacting a salt (II) with a nitrogen-containing compound, using at least two equivalents of nitrogen-containing compound per one equivalent of salt (II), said nitrogen-containing compound being chosen from amines, hydrazines and hydrazones.

24. A method for preparing a symmetrical streptocyanine (IV), characterized in that it consists in reacting a salt (II) with a nitrogen-containing compound, using at least two equivalents of nitrogen-containing compound per one equivalent of salt (II), said nitrogen-containing compound being chosen from guanidines, phosphaimines and amidines.

25. A method for preparing a macrocyclic dicationic compound (V), characterized in that it consists in reacting a salt (II) with a diamine H₂N-E-NH₂, using a salt (II)/diamine molar ratio of 1/1.

26. A method for preparing a heptacarbon hemicarboxonium salt (VI) or (VII), characterized in that it consists in reacting a salt (II) with a nitrogen-containing compound, using one equivalent of nitrogen-containing compound per one equivalent of salt (II), said nitrogen-containing compound being chosen from amines, hydrazines, and hydrazones in order to obtain a compound (VI) or from guanidines, phosphaimines and amidines in order to obtain compound (VII).

27. A method for preparing a disymmetrical cyanine (III), characterized in that it consists in reacting one equivalent of hemicarboxonium salt (VI) with one equivalent of a nitrogen-containing compound chosen from amines, hydrazines and hydrazones and different from that used for the preparation of said compound (VI) from the compound (II).

28. A method for preparing a disymmetrical cyanine (IV), characterized in that it consists in reacting one equivalent of hemicarboxonium salt (VII) with one equivalent of a nitrogen-containing compound chosen from guanidines, phosphaimines and amidines and different from that used for the preparation of said compound (VII) from compound (II).

29. A method for preparing a nonmacrocyclic polycationic compound as claimed in claim 10, characterized in that it consists in reacting n equivalents of salt (VI) with one equivalent of an amine having n primary or secondary amino groups, n≧2.

30. A method for labeling organic molecules, characterized in that it consists in using, as marker, a streptocyanine as claimed in one of claims 4 to 10.
